(19) United States
(12) Patent Application Publication
(10) Pub. No.: US 2003/0088168 A1
(43) Pub. Date: May 8, 2003

(54) Metered pump for a non-contact tonometer

(75) Inventor: David A. Luce, Clarence Center, NY (US)

Correspondence Address: Simpson, Simpson & Snyder, PLLC, 5555 Main Street, Williamsville, NY, 14221, US

(73) Assignee: Leica Microsystems Inc., Depew, NY (US)

(21) Appl. No.: 09/992,368

(22) Filed: Nov. 6, 2001

(51) Int. Cl. A61B 3/16

(52) U.S. Cl. 600/399; 600/401

(57) ABSTRACT

A metered fluid pump for a non-contact tonometer comprises a piston and a cylinder configured such that a plenum chamber defined thereby develops a massive leak through an exhaust port at a predetermined displacement position of the piston relative to the cylinder, thus reducing unnecessary impulse energy delivered to the eye that is a source of patient discomfort. A metering adjustment drive is provided for adjusting the piston and cylinder in a relative manner to meter the volume of fluid delivered before the leak develops, such that a suitable fluid pulse can be delivered to the eye.

### BACKGROUND OF THE INVENTION

The present invention relates generally to the field of ophthalmic instruments, and more particularly to non-contact tonometers for measuring intraocular pressure (IOP) of a patient's eye by deforming the cornea with a fluid pulse.

In the operation of non-contact tonometers, an increasing force fluid pulse is discharged through a tube directed at the eye to deform the cornea from a state of convexity, through an instantaneous state of “applanation” wherein a predetermined area of the cornea is flattened, to a state of concavity; the cornea is then allowed to return through a second state of applanation to its original convex state under natural forces. Opto-electronic means are used to monitor the corneal deformation and thereby determine the moment of applanation. Information related to the fluid pulse, such as a time interval required for the fluid pulse to achieve applanation or a plenum pressure associated with the fluid pulse at the time of applanation, is recorded and used as a correlate to IOP.

FIG. 1 shows a fluid pump 10 of the prior art for generating a fluid pulse in a non-contact tonometer. Fluid pump 10 comprises a cylinder 12 and a piston 14 axially displaceable relative to the cylinder for compressing air or another fluid within a plenum chamber 16 defined by the cylinder and piston. An electromechanical drive means 17, for example a solenoid or linear motor operatively connected to piston 14, applies force to displace the piston relative to cylinder 12. A discharge tube 18 is arranged in flow communication with plenum chamber 16 and is directed at the patient's cornea C for delivering the fluid pulse. While discharge tube 18 is shown in FIG. 1 as being in axial alignment with piston 14, it will be understood by those familiar with the art that piston 14 is very often not in axial alignment with discharge tube 18, particularly in where a rotary solenoid and coupling linkage are provided as drive means 17.

Since the fluid pulse directed at the patient's eye is very often a source of discomfort for the patient, it has been a recognized goal in the design of non-contact tonometers to deliver a “softer” fluid pulse. Stated differently, the goal has been to deliver only enough impulse energy to applanate cornea C, and to avoid delivering excess impulse energy that is unnecessary to the measurement process and only contributes to patient discomfort. Heretofore, efforts in pursuit of this goal have focused on controlling the energizing current supplied to drive means 18. For example, U.S. Pat. No. 5,279,300 teaches an approach wherein a predetermined shut-off time is assigned prior to beginning measurement for halting current supply to the drive means, and the current shut-off time is adjusted for subsequent measurements based on previous measurement results where a series of measurements are taken. As another example, U.S. Pat. No. 5,779,633 by the present applicant describes halting or reversing energizing current in response to a signal indicating the occurrence of applanation. A further example is found in U.S. Pat. No. 6,159,148 by applicant, which teaches controlling the energizing current supplied to the drive means to achieve a non-linear pressure increase in the fluid pump. Notably, none of the relevant prior art known to applicant has been directed to improving the physical configuration of the fluid pump itself.

As alluded to above with respect to U.S. Pat. No. 5,279,300, it is often preferable to take a series of measurements in sequence with respect to a single patient to obtain an accurate IOP reading. In these instances, a first measurement gives the practitioner an indication of the patient's IOP, and it is desirable to reduce the impulse energy delivered by the fluid pulse in subsequent measurements in accordance with the patient's IOP. However, reducing impulse energy by early shut-off of the energizing current, as taught in U.S. Pat. No. 5,279,300, may be detrimental to achieving an identifiable second applanation associated with outward “return” deformation of the cornea. This second applanation event is of value in practicing an improved measurement method as invented by applicant and disclosed in commonly-owned U.S. patent application Ser. No. 09/553,111 filed Apr. 20, 2000 entitled “NON-CONTACT TONOMETRY METHOD,” now U.S. Pat. No. ______.

### BRIEF SUMMARY OF THE INVENTION

Therefore, it is an object of the present invention to improve a non-contact tonometer by changing the configuration of a fluid pump of the non-contact tonometer to deliver less unnecessary impulse energy to the eye.

It is another object of the present invention to improve a non-contact tonometer by providing an exhaust port opening during a piston stroke of the fluid pump to allow superfluous pressurized fluid to escape rather than pass through a fluid discharge tube directed at the eye.

It is yet another object of the present invention to improve a non-contact tonometer by providing means for adjustably metering the volume of fluid delivered through a fluid discharge tube as a fluid pulse to applanate the cornea.

It is yet another object of the present invention to substantially reduce the “drawback” of eye fluids into a fluid discharge tube and fluid pump of a non-contact tonometer as a piston of the fluid pump is returned to its starting position and the fluid pump is recharged.

It is yet another object of the present invention to eliminate a shock wave normally associated with fluid pulse generation in a non-contact tonometer.

In accordance with the present invention, a non-contact tonometer of the type having a fluid pump communicating with a fluid discharge tube to generate and deliver a fluid pulse for deforming a cornea is characterized by a piston and cylinder configuration intentionally designed to develop a leak in a predetermined manner during the piston stroke, and by a metering adjustment drive working in conjunction with the piston and cylinder configuration to allow an operator to adjust the volume of fluid delivered through the fluid discharge tube before the leak develops.

In a first embodiment of the present invention, the cylinder of the fluid pump comprises telescopically arranged inner and outer tubular parts, and the inner part is provided with at least one axially elongated slot through its wall that begins at a distance from an open proximal end of the inner part and extends through a distal end of the inner part. Consequently, the inner and outer parts of the cylinder cooperate to define at least one flow channel running along the internal wall of the cylinder beginning at an intermediate axial location. The piston of the first embodiment includes a cylindrical plunger axially guided by the inner part of the cylinder. When a trailing edge of the cylindrical plunger passes the location where the flow channel or flow channels begin during a piston stroke, fluid within the plenum chamber defined by the piston and cylinder can escape via the flow channel or flow channels through the open proximal end of the inner part. A metering adjustment drive, such as a stepper motor, is connected to the inner part for adjusting the axial position of the inner part relative to the outer part, whereby the volume of fluid delivered before the leak develops can be controlled.

In a second embodiment of the invention, the cylinder is a one-piece tube having a circumferential arcuate slot through its wall at an intermediate axial location, and the piston includes a hollow cylindrical plunger open at its leading end and provided with an exhaust port through its wall such that a leak develops when the exhaust port overlaps with the slot of the cylinder during a piston stroke. Metering is achieved by an adjustment drive for altering the axial reference or start position of the piston relative to the cylinder.

A third embodiment of the present invention is similar to the second embodiment, except that the cylinder slot and plunger exhaust port are formed in a helical fashion to allow metering to be achieved through relative rotation between the piston and cylinder about the travel axis of the piston.

### BRIEF DESCRIPTION OF THE SEVERAL VIEWS OF THE DRAWINGS

The nature and mode of operation of the present invention will now be more fully described in the following detailed description of the invention taken with the accompanying drawing figures, in which:

FIG. 1 is a schematic view, partially in cross-section, showing a fluid pulse generating system of a non-contact tonometer in accordance with the prior art;

FIG. 2A is an exploded perspective view of a metered fluid pump for a non-contact tonometer formed in accordance with a first embodiment of the present invention;

FIG. 2B is a cross-sectional view of the metered fluid pump of FIG. 2A, shown in a charged condition;

FIG. 2C is a cross-sectional view of the metered fluid pump of FIG. 2A, shown in a discharged condition;

FIG. 3A is an exploded perspective view of a metered fluid pump for a non-contact tonometer formed in accordance with a second embodiment of the present invention;

FIG. 3B is a cross-sectional view of the metered fluid pump of FIG. 3A, shown in a charged condition;

FIG. 3C is a cross-sectional view of the metered fluid pump of FIG. 3A, shown in a discharged condition;

FIG. 4A is an exploded perspective view of a metered fluid pump for a non-contact tonometer formed in accordance with a third embodiment of the present invention;

FIG. 4B is a cross-sectional view of the metered fluid pump of FIG. 3A, shown in a charged condition;

FIG. 4C is a cross-sectional view of the metered fluid pump of FIG. 3A, shown in a discharged condition; and

FIG. 5 is an exemplary graph of plenum pressure versus time for a fluid pump according to the prior art and a fluid pump according to the present invention.

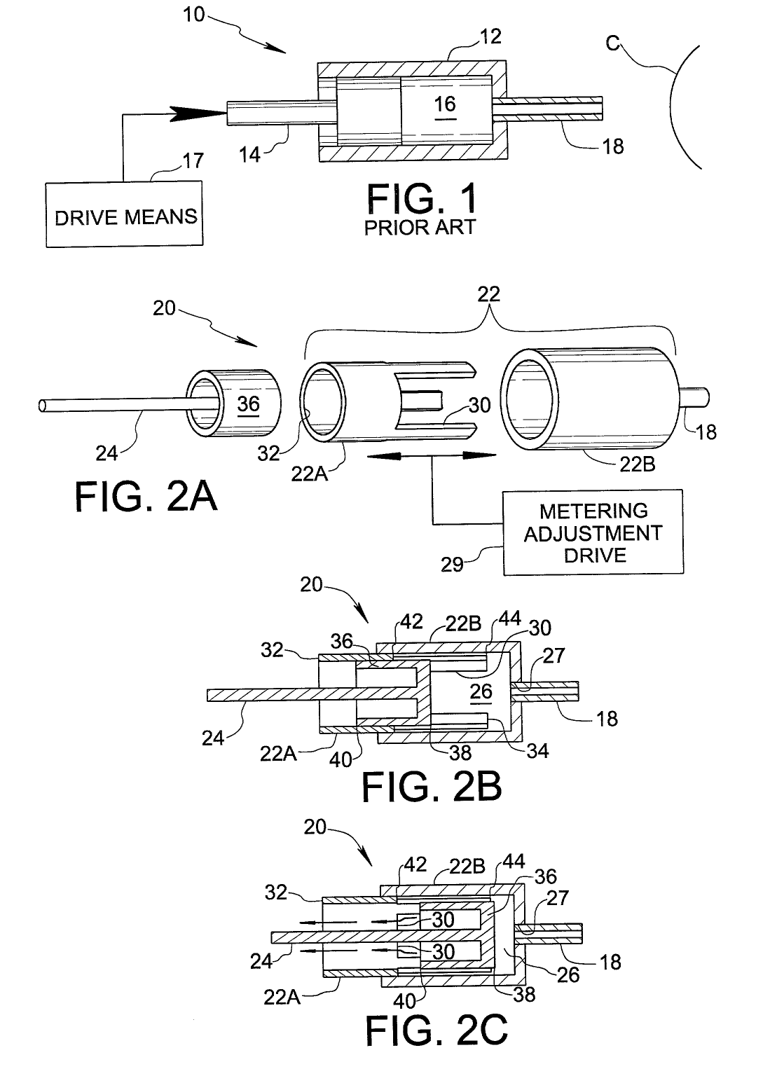

### DETAILED DESCRIPTION OF THE INVENTION

Attention is directed to FIGS. 2A-2C of the drawings, wherein a metered fluid pump formed in accordance with a first embodiment of the present invention is shown and designated generally by the reference numeral 20. Pump 20 comprises a cylinder 22 having an inner part 22A and an outer part 22B, and a piston 24 axially movable relative to cylinder 22 by suitable drive means (not shown) for compressing a fluid, commonly air, within a plenum chamber 26 defined by cylinder 22 and piston 24. Fluid discharge tube 18 is arranged in flow communication with plenum chamber 26 through a primary port 27 at a distal end of cylinder 22 for delivering a fluid pulse in a desired direction.

Inner part 22A and outer part 22B are telescopically arranged tubular bodies sized such that inner part 22A is received in close slidable fit within outer part 22A as shown. A metering adjustment drive 29, shown schematically in FIG. 2A, is operatively connected to inner part 22A for adjusting the axial position of inner part 22A relative to outer part 22B. Metering adjustment drive 29 may be any suitable drive means which allows the axial position of inner part 22A relative to outer part 22B to be controlled. For example, a stepper motor connected to inner part 22A would allow the axial position of inner part 22A to be precisely controlled and a step count would provide an indication of axial position. Alternatively, a motor that displaces inner part 22A in continuous fashion, rather than in discrete increments, could be used in combination with a position sensor arranged to sense the axial position of inner part 22A relative to outer part 22B and provide a signal representative of the axial position.

Inner part 22A is provided with at least one axially elongated slot 30 through its wall that begins at a distance from an open proximal end 32 of inner part 22A and extends through a distal end 34 of the inner part. In the embodiment shown, three slots 30 are provided at regular angular intervals about the axis of inner part 22A. Inner part 22A is sized to axially guide a plunger 36 of piston 24. Plunger 36 includes a leading edge 38 and a trailing edge 40. As can be understood from FIGS. 2B and 2C, each slot 30 cooperates with the inner wall of outer part 22B to define a flow channel extending in an axial direction from a first axial location 42 spaced from open proximal end 32 to a second axial location 44 spaced further from open proximal end 32. The unslotted proximal portion of inner part 22A forms a seal with outer part 22B.

FIG. 2B shows piston 24 in an initial reference position, wherein leading edge 38 of plunger 36 is between first location 42 and second location 44, and trailing edge 40 of plunger 36 is between open proximal end 32 and first location 42. Consequently, fluid in plenum chamber 26 can only escape through primary port 27 into discharge tube 18 as piston 24 begins its compression stroke. As piston 24 is driven in an axial direction during the compression stroke to generate a fluid pulse, trailing edge 40 eventually moves beyond first location 42, as shown in FIG. 2C. In accordance with the present invention, the axial distance between first location 42 and second location 44 is greater than the axial distance between leading edge 38 and trailing edge 40, whereby an alternate flow path opens from plenum chamber 26 through the flow channels defined by slots 30 and out open proximal end 32, to atmosphere. Thus, open proximal end 32 serves as an exhaust port for cylinder 22 and fluid pump 20 develops a massive leak through the exhaust port.

Metering adjustment drive 29 enables inner part 22A to be moved axially relative to outer part 22B prior to measuring patient IOP such that the exhaust port as defined by open proximal end 32 remains sealed until a selectable, predetermined volume of fluid has been forced from plenum chamber 26 through primary port 27 and into discharge tube 18, after which the exhaust port becomes unsealed to develop the aforementioned leak. When inner part 22A is inserted relatively deeply into outer part 22B, piston 24 must travel a greater distance until trailing edge 40 moves beyond first location 42, thereby delivering a greater volume of fluid through discharge tube 18 before the leak develops. Conversely, when inner part 22A is inserted to a lesser degree within outer part 22B, piston 24 travels a shorter distance before trailing edge 40 passes first location 42 and a smaller volume of fluid is forced through discharge tube 18 before the leak develops.

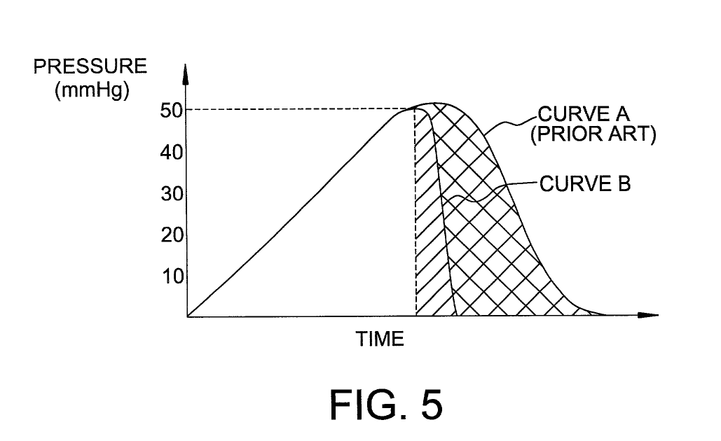

Fluid pump 20 offers several important benefits over the prior art. One such benefit can be understood with reference to FIG. 5 of the drawings, which presents a plot of plenum pressure versus time for a fluid pulse generated by a fluid pump of the prior art (CURVE A), and also for a fluid pulse generated by a fluid pump of the present invention (CURVE B). As can be seen, CURVE A falls off more gradually than CURVE B due to inertial effects of the piston and the need to dissipate energy stored in the compressed fluid, which must pass through the discharge tube. Assuming a peak pressure of 50 mmHg is desired, which would be sufficient to measure a very large percentage of the population, it can be seen from the entire shaded region (hatching and cross-hatching) under CURVE A that significant impulse is delivered to the patient's eye in excess of that necessary to achieve applanation, thereby contributing to patient discomfort. The sharp drop off exhibited by CURVE B due to the massive exhaust leak means that the overall duration of the fluid pulse and the excess impulse energy (see single direction hatching) delivered to the patient's eye are greatly reduced, thereby diminishing the nervous response of the patient and the perceived discomfort. This benefit becomes increasingly significant at higher IOP values, where excess impulse energy represented by area under the pressure-time curve after peak pressure grows much larger.

Another benefit offered by fluid pump 20 of the present invention is the ability to meter the fluid pulse so as not to exceed a chosen maximum or peak pressure value regardless of the amount of time associated with the compression stroke. Consequently, the improved fluid pump of the present invention is well suited for use in practicing a tonometric measurement method wherein two applanation events are observed, the first applanation event being associated with inward deformation of the cornea by the fluid pulse and the second applanation event being associated with outward or return deformation of the cornea under IOP as the fluid pulse dissipates. A more detailed description of this type of tonometric measurement method is found in the aforementioned U.S. patent application Ser. No. 09/553,111 filed Apr. 20, 2000, now U.S. Pat. No. ______, which is owned by the assignee of the present and incorporated by reference herein. The present invention does not preclude driving the piston in a manner that provides a longer stroke mode, which enhances observation of the inward and outward corneal applanations as distinct and comparable events. The piston can be driven in a manner which provides a linear pressure increase with time, as is well known in the art of non-contact tonometry. Alternatively, the piston can be driven in a manner which provides a non-linear pressure increase with time, as taught by commonly owned U.S. Pat. No. 6,159,148 issued Dec. 12, 2000 entitled “NON-CONTACT TONOMETER HAVING NONLINEAR PRESSURE RAMP,” such patent being hereby incorporated by reference into the present application.

An improved measurement methodology emerges as a result of the benefits discussed in the preceding paragraph. Where a series of sequential measurements are made with respect to a particular patient, the fluid pump of the present invention is easily metered based on one or more previous IOP measurements in the series to adjust the fluid volume forced through discharge tube 18. If an initial measurement made using a short stroke mode registers a normal IOP value, a subsequent measurement can be made after performing a metering adjustment of the fluid pump to reduce the impulse delivered by the fluid pulse in subsequent measurements. If an initial measurement made using a short stroke mode registers a relatively high IOP value, the piston can be driven under a long stroke mode, preferably in combination with fluid volume metering, to provide a more suitable detection signal for assessing effects of corneal thickness and corneal tissue properties on the IOP reading. In this way, a more accurate determination of IOP can be made.

A further benefit of the fluid pump of the present invention is that it eliminates a shock wave normally associated with fluid pulse generation, thereby obviating the need for a dashpot or damper in the fluid pump mechanism.

Yet another benefit of the fluid pump of the present invention is that the drawback of eye fluids into discharge tube 18 and the plenum chamber as the fluid pump recharged and the piston is returned to its original reference or start position is substantially reduced because the exhaust port acts as a secondary filling port through which air can pass for refilling the evacuated plenum chamber.

It will be appreciated that fluid pump 20 differs from the prior art in two important aspects: the provision of an exhaust flow leak at the end of the compression stroke and the ability to meter the volume of fluid delivered in the fluid pulse to the patient. Therefore, the present invention encompasses both aspects separately and in combination. This being the case, a simplified embodiment comprising a one-piece cylinder having flow channels formed in the inner wall thereof for developing the leak, without metering adjustment capability, is within the scope of the present invention. Likewise, another simplified embodiment comprising a two-piece cylinder having relatively adjustable inner and outer parts 22A and 22B for metering fluid volume, without provision of slots 30 for developing a leak, would also fall within the scope of the present invention.

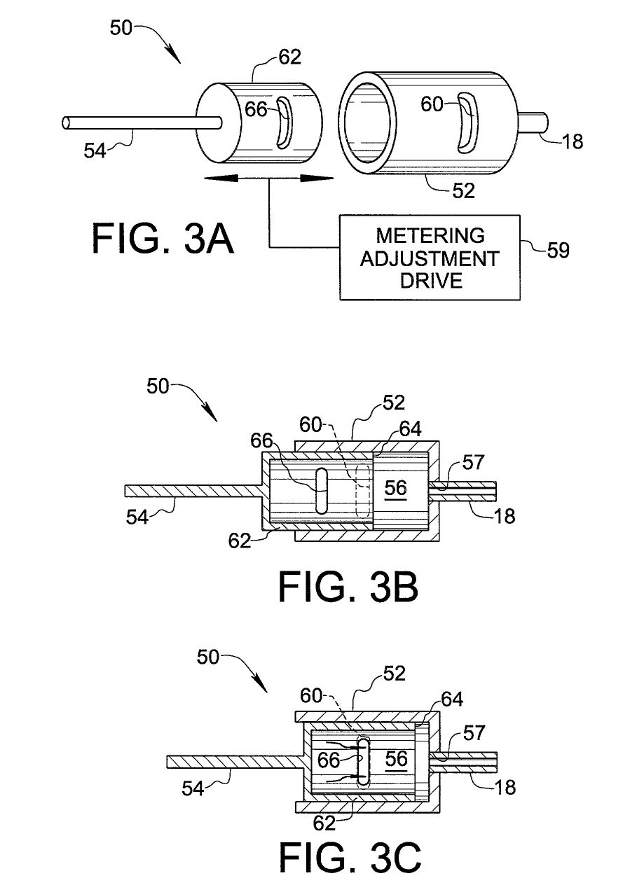

FIGS. 3A-3C illustrate a fluid pump 50 formed in accordance with a second embodiment of the present invention. Fluid pump 50 is generally similar to fluid pump 20 in that it comprises a cylinder 52 and a piston 54 axially movable relative to cylinder 52 by suitable drive means (not shown) for compressing fluid within a plenum chamber 56 defined by cylinder 52 and piston 54. Similar to the first embodiment, fluid discharge tube 18 is arranged in flow communication with plenum chamber 56 through a primary port 57 at a distal end of cylinder 52 for delivering a fluid pulse in a desired direction.

Cylinder 52 includes a circumferential arcuate slot 60 opening radially through its wall and serving as an exhaust port in the present embodiment. In addition, piston 54 includes a hollow plunger 62 having an open leading end 64 and a circumferential arcuate slot 66 opening radially through its wall. Slots 60 and 66 are positioned in cylinder 52 and plunger 62, respectively, and plunger 62 is elongated in an axial direction, such that the plunger wall seals slot 60 during an initial portion of the piston compression stroke, as depicted in FIG. 3B. However, as can be seen in FIG. 3C, once plunger slot 66 overlaps with cylinder slot 60, a leak develops and plenum chamber 56 is no longer in exclusive flow communication with discharge tube 18.

Metering of fluid volume in the second embodiment is carried out by a metering adjustment drive 59, shown in FIG. 3A, operatively connected to piston 54 for adjusting an initial axial reference position (start position) of piston 54 relative to cylinder 52. Metering adjustment drive 59 is analogous to metering adjustment drive 29 of the first embodiment, however metering adjustment drive 59 is linked to the piston instead of the cylinder.

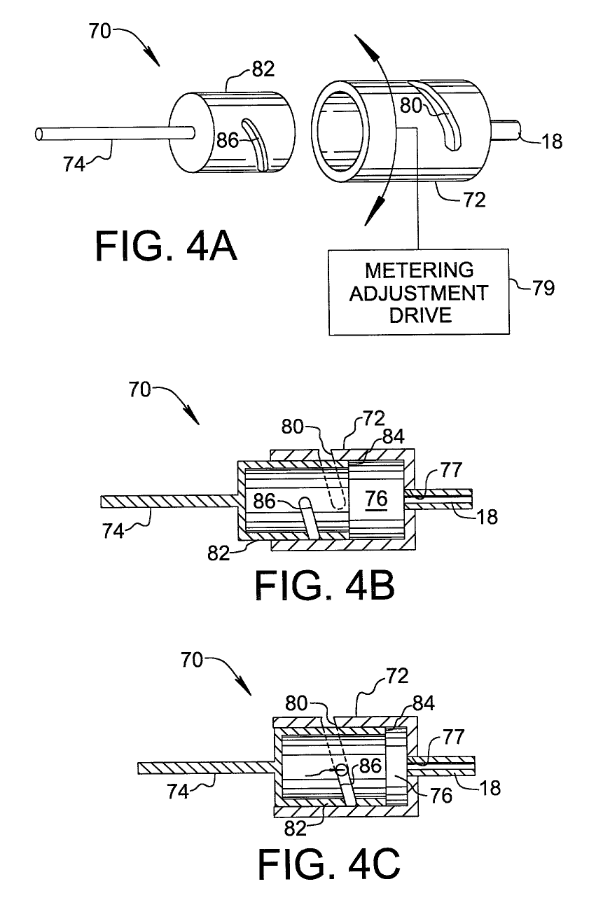

A fluid pump 70 formed in accordance with a third embodiment of the present invention is shown in FIGS. 4A-4C. Fluid pump 70 is similar to fluid pump 50 of the second embodiment in that fluid pump 70 comprises a cylinder 72 and a piston 74 axially movable relative to cylinder 72 by suitable drive means (not shown) for compressing fluid within a plenum chamber 76 defined by cylinder 72 and piston 74. Likewise, fluid discharge tube 18 is arranged in flow communication with plenum chamber 76 through a primary port 77 at a distal end of cylinder 72 for delivering a fluid pulse in a desired direction.

Cylinder 72 includes a helical slot 80 opening radially through its wall and serving as an exhaust port in the present embodiment. In addition, piston 74 includes a hollow plunger 82 having an open leading end 84 and a helical slot 86 opening radially through its wall. Slots 80 and 86 are positioned in cylinder 72 and plunger 82, respectively, and plunger 82 is elongated in an axial direction, such that the plunger wall seals slot 80 during an initial portion of the piston compression stroke, as depicted in FIG. 4B. However, as can be seen in FIG. 4C, once plunger slot 86 overlaps with cylinder slot 80, a leak develops and plenum chamber 56 is no longer in exclusive flow communication with discharge tube 18. It is noted here that although both cylinder slot 80 and plunger slot 86 are shown as being helical, one of these slots may simply be made to extend in an axial direction in any manner, including in a straight line.

Metering of fluid volume in the third embodiment is carried out by a metering adjustment drive 79, shown in FIG. 4A, operatively connected to piston 74 for adjusting an angular orientation of piston 74 relative to cylinder 72 about the axis of displacement of the piston. Metering adjustment drive 79 of the third embodiment is analogous to metering adjustment drives 29 and 59 of the first and second embodiments.

What is claimed is:

1. In a non-contact tonometer having a fluid pump communicating with a fluid discharge tube to generate and deliver a fluid pulse for deforming a cornea, wherein said fluid pump comprises a cylinder and piston displaceable relative to said cylinder for compressing fluid within a plenum chamber defined by said piston and said cylinder, the improvement comprising:
said piston and said cylinder being configured such that said plenum chamber transitions from being in exclusive communication with said fluid discharge tube to being in non-exclusive communication with said fluid discharge tube as said piston moves beyond a predetermined displacement position relative to said cylinder.

2. The improvement according to claim 1, wherein said plenum chamber is in communication with atmosphere when said piston is beyond said predetermined displacement position.

3. In a non-contact tonometer having a fluid pump communicating with a fluid discharge tube to generate and deliver a fluid pulse for deforming a cornea, wherein said fluid pump comprises a cylinder and piston displaceable relative to said cylinder for compressing fluid within a plenum chamber defined by said piston and said cylinder, the improvement comprising:
said cylinder having an inner tubular part and an outer tubular part for receiving said inner tubular part, wherein a volume of fluid delivered in said fluid pulse is metered by axially adjusting said inner and outer tubular parts relative to one another prior to generating said fluid pulse.

4. The improvement according to claim 3, further comprising a metering adjustment drive connected to said inner tubular part for axially moving said inner tubular part relative to said outer tubular part.

5. A fluid pump system for a non-contact tonometer, said fluid pump system comprising:
a fluid discharge tube;
a cylinder having a primary port and an exhaust port; and
a piston displaceable along an axis relative to said cylinder from an initial reference position, said piston and said cylinder defining a plenum chamber in communication with said fluid discharge tube through said primary port;
wherein said exhaust port is sealed until said piston has forced a predetermined volume of fluid from said plenum chamber through said primary port, and thereafter said exhaust port becomes unsealed such that said plenum chamber develops a leak through said exhaust port.

6. The fluid pump system according to claim 5, wherein said cylinder comprises telescopically arranged inner and outer tubular parts, and said predetermined volume of fluid is metered by axially adjusting said inner and outer parts relative to one another.

7. The fluid pump system according to claim 6, further comprising an automatic drive mechanism arranged to axially adjust said inner and outer parts relative to one another.

8. The fluid pump system according to claim 7, wherein said automatic drive mechanism is operatively connected to said inner part.

9. The fluid pump system according to claim 5, wherein said piston includes a plunger having a leading edge and a trailing edge, and said cylinder comprises a tubular body open at a proximal end thereof, said tubular body being sized to guide said plunger in an axial direction of said tubular body and having at least one internal flow channel extending in said axial direction from a first location spaced from said proximal end of said tubular body to a second location spaced further from said proximal end of said tubular body, the axial distance between said first location and said second location being greater than the axial distance between said leading edge and said trailing edge of said plunger;
whereby said open proximal end of said tubular body acts as said exhaust port when said trailing edge of said plunger moves beyond said first location.

10. The fluid pump system according to claim 6, wherein said piston includes a plunger having a leading edge and a trailing edge, and said inner part of said cylinder is open at a proximal end thereof and sized to guide said plunger in an axial direction of thereof, said inner part having at least one internal flow channel extending in said axial direction from a first location spaced from said proximal end of said inner part to a second location spaced further from said proximal end of said inner part, the axial distance between said first location and said second location being greater than the axial distance between said leading edge and said trailing edge of said plunger;
whereby said open proximal end of said inner part acts as said exhaust port when said trailing edge of said plunger moves beyond said first location.

11. The fluid pump system according to claim 10, wherein said inner part includes said open proximal end, said outer part includes said primary port, and said at least one flow channel is defined by a slot in said inner part.

12. The fluid pump system according to claim 5, wherein said exhaust port extends in a radial direction through said cylinder, and said piston includes a hollow plunger open at its front end and elongated in said axial direction to seal said exhaust port, said plunger having an escape passage extending in a radial direction therethrough, whereby said exhaust port becomes unsealed when said escape passage of said plunger overlaps with said exhaust port.

13. The fluid pump system according to claim 12, wherein said exhaust port is a circumferential arcuate slot in said cylinder and said predetermined volume of fluid is metered by adjusting said initial reference position.

14. The fluid pump system according to claim 12, wherein said escape passage is a circumferential arcuate slot in said plunger and said predetermined volume of fluid is metered by adjusting said initial reference position.

15. The fluid pump system according to claim 12, wherein said exhaust port is a circumferential arcuate slot in said cylinder, said escape passage is a circumferential arcuate slot in said plunger, and said predetermined volume of fluid is metered by adjusting said initial reference position.

16. The fluid pump system according to claim 12, wherein said exhaust port is a helical slot in said cylinder and said predetermined volume of fluid is metered by adjusting the angular orientation of said piston relative to said cylinder about said axis of displacement.

17. The fluid pump system according to claim 12, wherein said escape passage is a helical slot in said plunger and said predetermined volume of fluid is metered by adjusting the angular orientation of said piston relative to said cylinder about said axis of displacement.

18. The fluid pump system according to claim 12, wherein said exhaust port is a helical slot in said cylinder, said escape passage is a helical slot in said plunger, and said predetermined volume of fluid is metered by adjusting the angular orientation of said piston relative to said cylinder about said axis of displacement.

19. In a method of measuring intraocular pressure of an eye by taking sequential measurements using a non-contact tonometer having a fluid pump for generating a fluid pulse to deform the cornea of said eye, the improvement comprising the step of:
metering a volume of fluid used to generate said fluid pulse based on a prior measurement result.

20. The improvement according to claim 19, wherein said volume of fluid is metered by causing a leak to develop in said fluid pump at a chosen point during generation of said fluid pulse.
